(19) United States
(12) Patent Application Publication
(10) Pub. No.: US 2008/0306420 A1
(43) Pub. Date: Dec. 11, 2008

(54) COMPRESSION DEVICE WITH INDEPENDENTLY MOVEABLE INFLATABLE MEMBER

(75) Inventor: Mark A. Vess, Hanson, MA (US)

Correspondence Address: TYCO HEALTHCARE - EDWARD S. JARMOLOWICZ, 15 HAMPSHIRE STREET, MANSFIELD, MA, 02048, US

(73) Assignee: Tyco Healthcare Group LP, Mansfield, MA (US)

(21) Appl. No.: 12/059,828

(22) Filed: Mar. 31, 2008

Related U.S. Application Data
(63) Continuation-in-part of application No. 11/761,212, filed on Jun. 11, 2007.
Application No. 11/761,212 is a continuation-in-part of application No. 11/760,321, filed on Jun. 8, 2007, now abandoned.

(51) Int. Cl. A61F 5/00

(52) U.S. Cl. 602/13; 602/27; 602/23

(57) ABSTRACT

A compression device includes an inflatable member mounted on a mounting layer. The device may include a second layer secured to the mounting layer to form an envelope member in which the inflatable member is disposed. The second layer may be a non-mounting layer, to which the inflatable member is not secured, or another mounting layer, to which the inflatable member is secured. A securement mechanism by which the inflatable member is secured to either the mounting layer or both the mounting layer and the second layer allows the inflatable member to move independently relative to either the second layer or to both the second layer and the mounting layer.

This application is a continuation-in-part of U.S. Ser. No. 11/761,212, filed Jun. 11, 2007 which is a continuation-in-part of U.S. Ser. No. 11/760,321, filed Jun. 8, 2007, the entire contents of which are incorporated herein by reference.

### BACKGROUND

The present disclosure relates generally to a compression device. In particular, the present disclosure relates to a compression device configured for applying compressive forces to a portion of a wearer's anatomy.

Compression devices for applying compressive forces to a selected area of a wearer's anatomy are generally employed to improve blood flow in the selected area. Compression devices that provide intermittent pulses of a compressed fluid (i.e. air) to inflate at least one inflatable chamber in a cuff are particularly useful. This cyclic application of pressure provides a non-invasive method of prophylaxis to reduce the incidence of deep vein thrombosis (DVT), and the like. These compression devices find particular use during surgery on patients with high-risk conditions such as obesity, advanced age, malignancy, or prior thromboembolism. Patients who develop this condition often have swelling (edema) and tissue breakdown (venous stasis ulcer) in the lower leg. When a DVT occurs, the valves that are located within the veins of the leg can be damaged, which in turn can cause stasis and high pressure in the veins of the lower leg.

Generally, these compression devices are fluidly coupled to a source of pressurized fluid by one or more air tubes. Additionally, each compression device includes a flexible shell having one or more inflatable members disposed therein. The compression device is placed around the patient's foot or other selected portion whereupon a pressurized fluid is delivered into the inflatable member creating pressure at the part or parts of the body in contact with the inflatable member.

Compression cuffs adapted for use with a patient's foot may be combined with one or more additional compression cuffs that are disposed on portions of a patient's leg for improving the treatment regimen. In general, each of the additional compression cuffs includes a plurality of separate inflatable chambers that are progressively arranged along a longitudinal axis of the cuff from a lower portion to an upper portion of the limb. A pressure source, e.g. a controller, is provided for intermittently forming a pressure pulse within these inflatable chambers from a source of pressurized fluid during periodic compression cycles. The compression cuffs provide a pressure gradient along the patient's limbs during these compression cycles which progressively decreases from the lower portion to the upper portion of the limb (e.g. from the ankle to the thigh).

Compression cuffs that are adapted for use with a patient's foot generally include a heel strap with a tab portion that is adapted to fit around a portion of the patient's heel. This arrangement allows the compression cuff to be releasably attached to the patient's foot.

Examples of compression cuffs are disclosed in U.S. Pat. Nos. 4,013,069 and 4,030,488 to Hasty, U.S. Pat. Nos. 4,029,087 and 5,795,312 to Dye, U.S. Pat. No. 5,626,556 to Tobler et al., and U.S. patent application Ser. No. 11/761,212 to Meyer et al., all of which are currently owned by Tyco Healthcare Group LP and are incorporated by reference herein in their entireties. Other examples of compression cuffs are disclosed in U.S. Pat. Nos. 4,696,289 to Gardner et al. and 5,989,204 to Lina. An example of compression treatment method is disclosed in U.S. Pat. No. 6,231,532 to Watson et al., which is currently owned by Tyco Healthcare Group LP, the contents of which are hereby incorporated by reference herein in their entirety.

Some prior art devices are bulky and may irritate portions of the limb undergoing treatment which may increase patient discomfort and may increase the possibility that the patient may not complete the treatment regimen.

### SUMMARY

In one aspect of the present invention, a compression device for applying compressive force to a part of a wearer's body generally comprises an envelope comprising a mounting layer and a non-mounting layer disposed in generally opposing relationship to one another and being secured to one another to define an interior space within a periphery of the envelope. An inflatable member is disposed within the spaced defined by the interior space of the envelope. The inflatable member comprises layers in generally opposing relationship with one another. The layers are secured to one another to define an inflatable chamber. At least one of the layers extends beyond the periphery of the chamber to define a perimeter margin of the inflatable member. The perimeter margin of the inflatable member is secured to the mounting layer of the envelope member to mount the inflatable member on the mounting layer. The inflatable member is free from direct securement to the non-mounting layer to allow the inflatable member to move independently relative to the non-mounting layer.

In another aspect, a compression device for applying compressive force to a part of a wearer's body generally comprises at least one inflatable member comprising inner and outer layers each having respective inner and outer faces. The inner face of the outer layer is in a generally opposing relationship with the outer face of the inner layer. The inner and outer layers are secured to one another to define an inflatable chamber. A mounting layer is in generally opposing relationship with one of the outer face of the outer layer and the inner face of the inner layer. A fastening mechanism mounts the at least one inflatable member to the mounting layer while allowing the at least one inflatable member to move independently relative to the mounting layer.

Other features will be in part apparent and in part pointed out hereinafter. Various refinements exist of the features noted in relation to the above-mentioned aspects of the present invention. Further features may also be incorporated in the above-mentioned aspects of the present invention as well. These refinements and additional features may exist individually or in any combination. For instance, various features discussed below in relation to any of the illustrated embodiments of the present invention may be incorporated into any of the above-described aspects.

### BRIEF DESCRIPTION OF THE DRAWINGS

The features of the presently disclosed compression device will become more readily apparent by referring to the following detailed description of embodiments, which are described herein below with reference to the drawings, wherein:

FIG. 1 is an exploded perspective of a first embodiment of a compression foot cuff in accordance with the present disclosure;

FIG. 2 is a top view of the foot cuff illustrated in FIG. 1 with a central part of an inner layer of the device broken away;

FIG. 3 is a top view of the foot cuff of FIG. 1, illustrating the outline of a foot of a wearer disposed thereon;

FIG. 4 is a fragmentary section of the foot cuff of FIG. 1 taken along the line 4-4 in FIG. 2;

FIG. 5 is a top view of a compression leg sleeve with an inner layer of the sleeve partially broken away;

FIG. 6 is a top view of another embodiment of a foot cuff with an inner layer of the cuff removed;

FIG. 7 is a fragmentary section of the foot cuff of FIG. 6 taken along the line 7-7 in FIG. 6;

### DETAILED DESCRIPTION

With reference to the drawing figures, in which like references numerals identify identical or corresponding elements, various embodiments of the presently disclosed compression apparatus will now be described in detail.

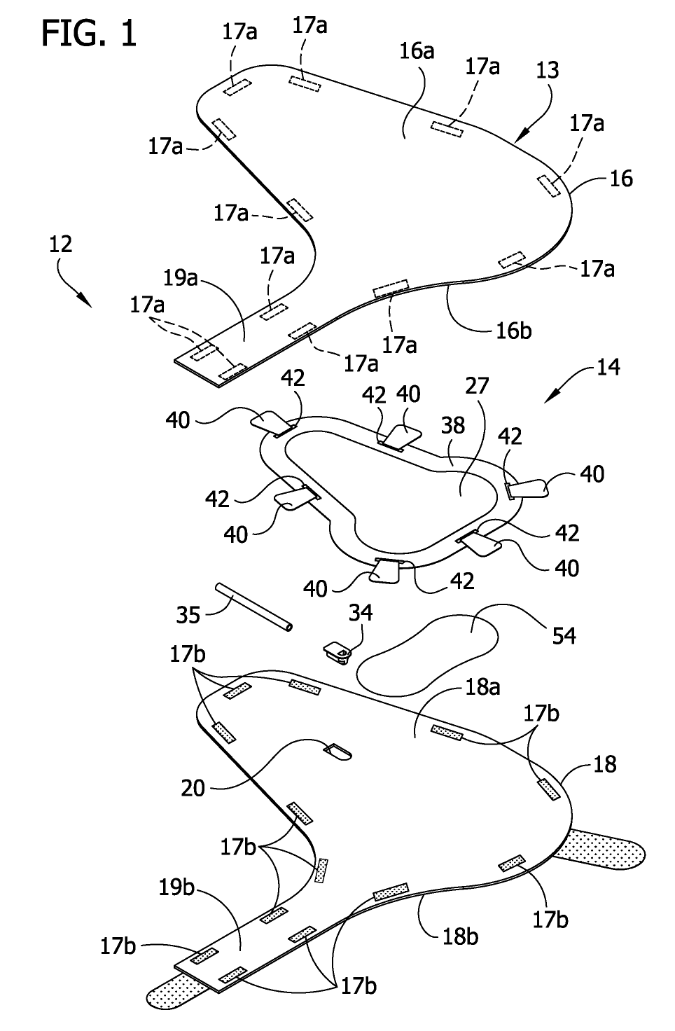

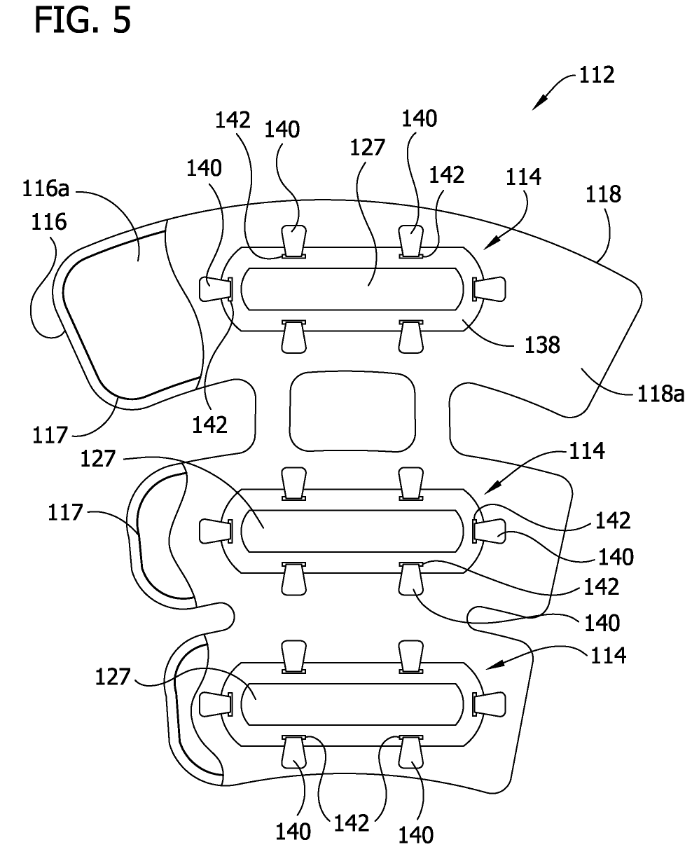

With initial reference to FIGS. 1-4, a first embodiment of a compression device in accordance with the present disclosure is illustrated and is designated generally as foot cuff 12. The foot cuff is adapted for use in a compression therapy system for applying compressive pressure to a foot of a wearer. The illustrated foot cuff 12 is configured and dimensioned for disposing about the right foot of the subject. It is understood that the foot cuff 12 may take on other configurations within the scope of the present invention. It is also understood that other types of compression devices besides foot cuffs, including but not limited to leg compression sleeves (FIG. 5), arm compression sleeves and other devices are within the scope of the present invention.

As shown best in FIG. 1, foot cuff 12 includes an envelope, generally indicated at 13, substantially enveloping or enclosing a bladder or an inflatable member 14 therein. The envelope 13 includes an inner contact layer 16 and an outer layer 18 each having inner and outer faces, 16a, 16b and 18a, 18b, respectively. As used herein, the terms “inner” and “outer” indicate relative positions of respective components and surfaces with respect to the skin of the wearer's body part when the compression device is secured to the body part, and as such, an “inner” component or surface is more adjacent to the skin of the body part than an “outer” component or surface. The inner contact layer 16 of the envelope 13 overlies the outer layer 18 so that the inner face 18a of the outer layer and the outer face 16b of the contact layer are in a generally opposing relationship. The outer layer 18 and the contact layer 16 are joined to one another generally adjacent to corresponding perimeters of the layers. The contact layer 16 and the outer layer 18 are releasably secured to one another by hook and loop fasteners 17a, 17b disposed on respective layers so that the envelope 13 is repeatedly openable and closable to allow access to the inflatable member 14 in the interior space of the envelope. It is understood that the contact layer 16 and the outer layer 18 may be secured to one another in other suitable ways. The contact layer 16 and the outer layer 18 may be fixedly secured to one another, such as by heat welding, adhesives, sewing or other suitable ways. The contact layer 16 and the outer layer 18 define an interior space for receiving and substantially enclosing the inflatable member 14 therein.

Contact layer 16 and outer layer 18 of the envelope 13 include strap portions 19a and 19b respectively. Strap portions 19a, 19b have a longitudinally projecting configuration for wrapping about a portion of the foot adjacent to the ankle. Strap portions 19a, 19b can be sewn, RF welded, or sonic welded. However, in the illustrated embodiments, the strap portions 19a, 19b are formed as one piece with the contact layer 16 and outer layer 18, respectively.

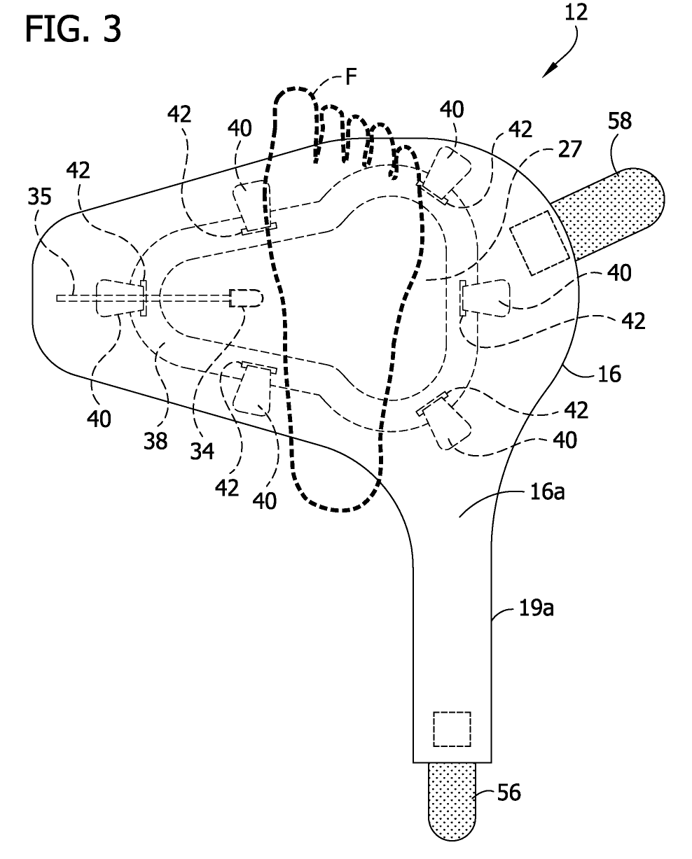

Contact layer 16 of the envelope 13 is adapted for contacting the bottom portion of the foot F (FIG. 3) and providing a fabric on fabric slide with inflatable member 14, in accordance with the present disclosure. Contact layer 16 is in one embodiment fabricated from a chemically treated material, with wicking ability, for wicking away moisture from the skin. In one embodiment, contact layer 16 includes a mesh-like fabric capable of wicking moisture away from the patient's skin. Furthermore, the contact layer 16 can be faced with a soft material toward the treatment surface of the patient. For example, the material can be a thin layer of open celled porous foam, napped cloth, or a layer of vapor permeable cloth permeable. For reasons explained below, the contact layer 16 may be coated with or otherwise include a low-friction layer (such as Teflon®) opposing the inflatable bladder. It is understood that the cuff 12 may not include a contact layer within the scope of the present invention.

Outer layer 18 of the envelope 13 includes an opening 20 for permitting a pressurized fluid inlet passage therethrough. Outer layer 18 is configured for providing the attachment surface for a hook and loop feature of cuff 12, as will be described in detail herein below. Moreover, the outer layer 18 provides a soft material for cushioning effect against the top portion of the feet and may be fabricated from similar materials as contact layer 16 and in similar dimensions therewith for corresponding geometry. Alternatively, outer layer 18 may be fabricated from a laminated material, such as, for example, sontara fabric, open cell urethane foam, or loop fabric. It is understood that the cuff 12 may not include an outer layer within the scope of the present invention.

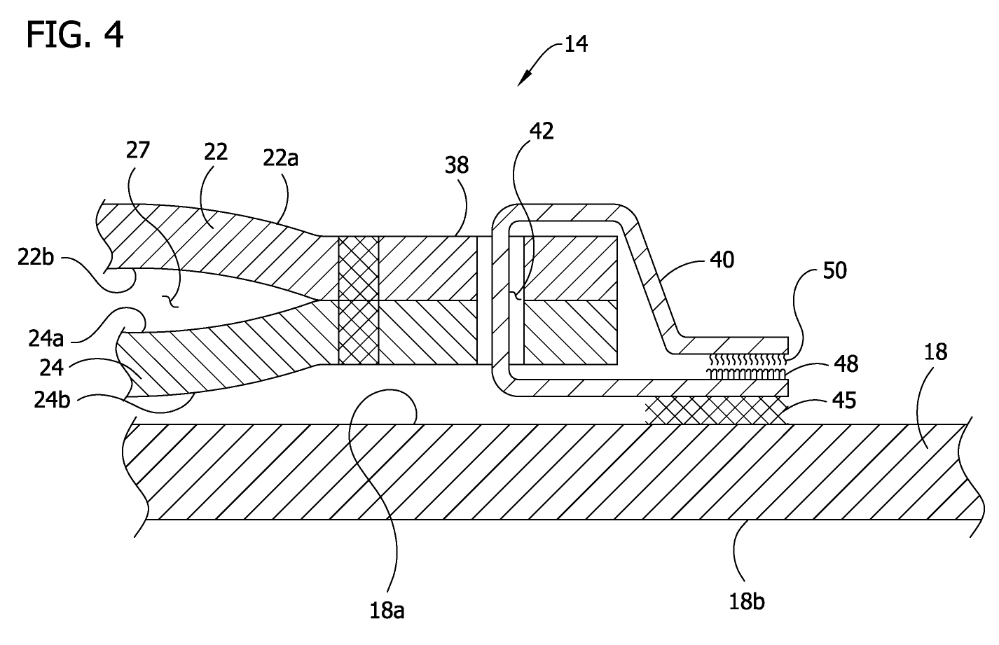

With particular reference to FIG. 3, inflatable member 14 is configured for positioning against the bottom portion of the foot. Referring to FIG. 4, inflatable member 14 includes inner and outer layers 22, 24 of air impermeable material (e.g., PVC) joined together in a suitable manner at their peripheries to define a single inflatable chamber 27. The chamber may be formed by radio frequency (RF) welding. Alternatively, the sealing line 26 may be sewn, formed by adhesive, heat sealing, etc. It is understood that the inflatable member 14 can include more than one inflatable chamber 27 within the scope of the present invention. For reasons explained below, the exterior surface and in particular the inner layer 22 of the inflatable member 14 may be coated with or otherwise include a low-friction layer (such as Teflon®) opposing the contact layer 16 in the illustrated embodiment. The inflatable chamber 27 of the inflatable member 14 is adapted for receiving and retaining a pressurized fluid (e.g. air) for exerting compressive pressure to the foot during successive pressure applying cycles. The inflatable chamber 27 has an inlet member 34 and a tube 35 connected to the inlet member for air or fluid to be introduced into the chamber during the start of a compression cycle and to be exhausted to end the compression cycle. The inlet member 34 of the illustrated embodiment is a plastic component that is secured such as by heat welding or other means to the inflatable member 14. It is understood that other ways of introducing air or fluid into the chamber 27 is within the scope of the invention.

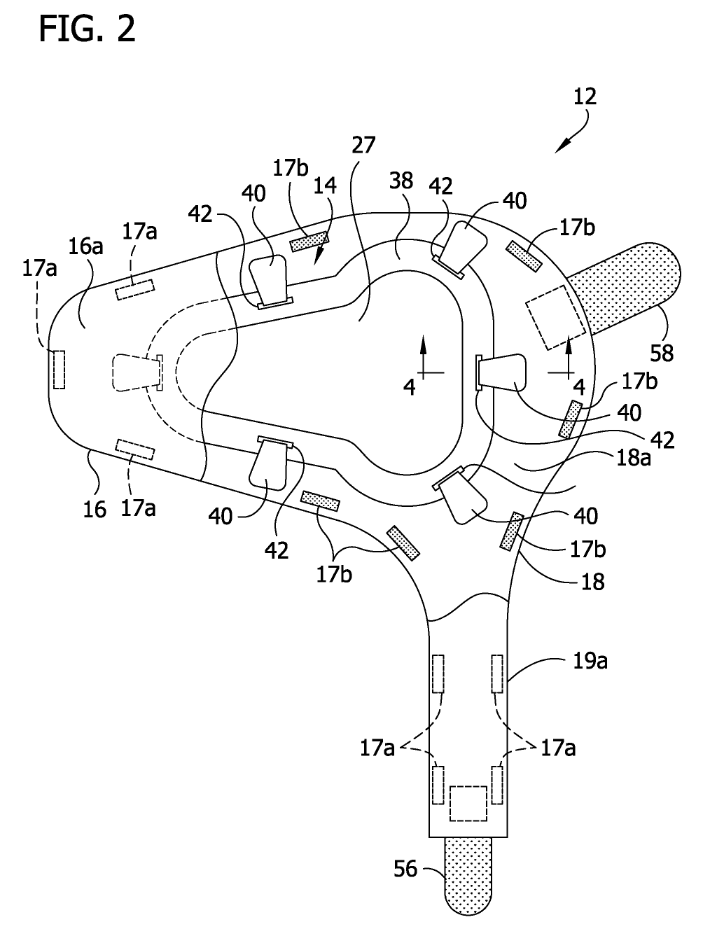

Referring to FIGS. 2-4, the inflatable member 14 includes a perimeter margin 38 extending outside or beyond the periphery of the chamber 27 to the perimeter edge of the inflatable member. As explained below, the perimeter margin 38 of the inflatable member 14 is secured to the outer layer 18 of the envelope 13, and the inflatable member 14 is free from securement to the inner contact layer 16. Accordingly, in the illustrated embodiment the outer layer 18 constitutes a “mounting layer” because the perimeter margin 38 of the inflatable member is secured to the outer layer; the inner contact layer 16 constitutes a “non-mounting layer” because the inflatable member 14 is not secured to the inner contact layer. Through this configuration, the inflatable member 14 is allowed to shift or move independently relative to the inner contact layer 16 (i.e., the non-mounting layer) because the inflatable member is secured to or mounted on only the outer layer (i.e., the mounting layer). In an embodiment where the contact layer 16 and/or the inflatable member 14 is coated with or otherwise includes a low-friction layer (such as Teflon®) so that the low-friction layer is at an interface of the contact layer and inflatable member, this low-friction layer further aids in allowing the inflatable member to move independently relative to the contact layer by reducing any frictional forces between the inflatable member and the contact layer and allowing the inflatable member to readily slide along the contact layer. It is understood that the cuff 12 may be configured so that the inner contact layer 16 constitutes the mounting layer and the outer layer 18 constitutes the non-mounting layer. As will be explained below, it is also envisioned that the cuff 12 may be configured so that inflatable member 14 is allowed to shift or move independently relative to the non-mounting layer (e.g., the inner contact layer 16) as well as the mounting layer (e.g., the outer layer 18).

Referring to FIGS. 1-4, a plurality loop components 40 secured to the outer layer 18 of the envelope 13 extend through a plurality of slits 42 (broadly, securement holes) in the perimeter margin 38 of the inflatable member 14 to secure the inflatable member to the outer layer. The slits 42 are spaced apart from one another around the periphery of the chamber sealing line 26 of the inflatable member 14. In the illustrated embodiment, the perimeter margin 38 of the inflatable member 14 encompasses both the perimeter margin of the inner inflatable layer 16 and the perimeter margin of the outer inflatable layer 18. As such, the slits 42 extend through both the outer inflatable layer 18 and the inner inflatable layer 16. It is contemplated, however, that only one of the inflatable layers 16, 18 may make up the perimeter margin 38 of the inflatable member 14, and the slits 42 may extend through only one of the inflatable layers. It is envisioned that the slits 42 could be reinforced by grommets (not shown) or in another suitable manner. In the illustrated embodiment and referring to FIG. 4, each loop component 40 is formed from a flexible strap (denoted by the same reference numeral 40) configured generally in the shape of a loop. The strap 40 has a first end margin secured to the inner face 18a of the outer layer 18. In one example given without limitation, the first end margin of the strap 40 may be fixedly secured to the outer layer 18 by sewing, spot welding, adhesive or other suitable means. In another example given without limitation, the first end margin of the strap 40 may be releasably secured to the outer layer 18 by hook and loop fasteners, snap-fasteners or other suitable means. In the illustrated embodiment the first end margin of the strap 40 is secured to the outer layer 18 by adhesive 45. It is understood that other components or structure other than a flexible strap may be used to form the loop.

Each strap 40 extends between the inflatable member 14 and the outer cover 18 and enters the slit 42 in the perimeter margin 38. Each strap extends through the corresponding slit 42 and loops back toward its first end margin. A second end margin of the strap 40 is releasably secured to the first end margin to close the loop and to allow the loop to be openable. As a result of the loop 40 being openable and the envelope 13 being openable by way of the hook and loop fasteners 17a, 17b on the respective contact layer 16 and outer layer 18, the inflatable member 14 can be removed from the cuff 12 and reused in another cuff. The inflatable member 14 can be readily removed from the cuff 12 and reused in another cuff of corresponding construction. It is understood that the second end margin of the strap 40 may be secured to the outer cover 18 or to another component or surface other than the first end margin of the strap within the scope of the invention. In the illustrated embodiment, the first end margin of the strap 40 includes a hook fastener 48 (broadly, a first fastener) and the second end margin of the strap includes a loop fastener 50 (broadly, a second fastener) that is releasably securable or mateable to the hook fastener at the first end margin. The respective locations of the hook and loop fasteners 48, 50, respectively, may be reversed within the scope of the invention. Moreover, the respective end margins of the straps may include other types of releasable, mateable fasteners, including for example, snap-fasteners. Moreover still, the first and second end margins of each strap 40 may be non-releasably or fixedly attached to one another, such as by heat welding or adhesive or sewing or other suitable means, for example, so that the loop cannot be opened. Other ways of providing a non-openable loop component are within the scope of the present invention.

The inflatable member 14 of the illustrated embodiment is secured to the outer layer 18 of the envelope 13 solely by the loop components 40 and the inflatable chamber 27 is free from direct securement to the outer layer. As stated briefly above, the cuff 12 may be configured so that the inflatable member 14 is movable independently relative to only the non-mounting layer and not to the mounting layer. Accordingly, in the illustrated embodiment the loop components 40 may be radially non-stretchable, which means that in the illustrated embodiment where the loop components are formed from straps, the straps would be non-stretchable along their lengths. In this embodiment, the loop components 40 would hold the inflatable member 14 generally fixedly with respect to the outer layer 18 so that the inflatable member 14 does not substantially move or shift relative to the outer layer but does shift or move with respect to the inner contact layer 16.

As also stated briefly above, the cuff 12 may be configured so that the inflatable bladder is movable independently relative to both the non-mounting layer and the mounting layer. Accordingly, in the illustrated embodiment, the loop components 40 may be “loosely fitted” to the inflatable member 14 to allow the inflatable member 14 to move or shift relative to the outer layer 18. In other words, each loop component 40 may have a relatively large radius to allow the inflatable member 14 to shift in all directions along the inner surface 18a of the outer layer 18. In another embodiment, the loop components 40 may be radially and elastically stretchable, which means that in the illustrated embodiment, where the loop components are formed from straps, the straps would be resiliently or elastically stretchable along their lengths. In this embodiment, the loop components 40 resiliently stretch, allowing the inflatable member to move independent of the outer layer 18. In yet another embodiment, the loop components may be non-stretchable and “tightly fitted” to the inflatable member. In this embodiment, the inflatable member 14 may shift independently of the outer layer 18 because the inflatable member 14, and in particular the perimeter margin 38 of the inflatable member, is stretchable, and preferably resiliently stretchable. In yet still another embodiment, the slits 42 or holes in the perimeter margin 38 of the inflatable member are sized and shaped to allow the inflatable member to shift or move independent of the outer layer 18. In this embodiment, both the inflatable member 14 and the loop component 40 may be non-stretchable and the slits 42 may be sized and shaped to allow movement of the inflatable member relative to the loop components. Substitutions and/or combinations of the features of any of the above embodiments are within the scope of the invention. Other ways of making the inflatable member 14 movable relative to and independent of the outer layer 18 and the inner layer 16 are within the scope of the present invention.

It is envisioned that in certain embodiments (not shown), the loop components 40 may be secured to both the inner contact layer 16 and the outer layer 18, and the inflatable member 14 may be moveable independently with respect to both the inner contact layer and the outer layer. In effect, both the inner contact layer 16 and the outer layer 18 would be considered “mounting layers”. For example and without limitation, where the loop components 40 are radially stretchable (e.g., elastically stretchable), the first end margin of the strap (or other member) may be secured to the outer layer 18 and the second end margin of the strap may be secured to the inner layer 16. Because the loop components are stretchable, the inflatable member 14 is moveable independently relative to both the inner contact layer 16 and the outer layer 18 of the envelope 13. Other ways of making the inflatable member 14 moveable independently relative to both layers 16, 18 of the envelope 13, are within the scope of the invention. Substitutions and/or combinations of the features of any of the above embodiments are within the scope of the invention.

It is contemplated that the inflatable member 14 may be free from securement to the outer layer 18, and the loop components 40 may be used to secure the perimeter margin 38 of the inflatable member to the inner layer 16 instead of to the outer layer. It is understood that this embodiment (not shown) would function in substantially the same manner as the embodiment where the loop components 40 secure the perimeter margin 38 of the inflatable member 14 to the outer layer 18.

Referring again to FIG. 1, the cuff 12 includes a sole member 54. It is understood that the cuff may not have a sole member within the scope of the present invention. The sole member 54 is disposed between the inflatable member 14 and the outer layer 18 as illustrated in FIG. 1, although it may be situated in other locations within the scope of the invention. The sole member 54 is generally shaped according to contours of the sole of the foot. The sole member 54 is made of a rigid material, for example, metal, polymers, ceramics or composite-type materials. As is generally known in the art, the sole member 54 allows compression to be more directly applied to the foot.

The sole member 54 may be attached to the inflatable member 14, such as by a strip of double stick adhesive tape. Alternatively or in addition, the position of the sole member 39 relative to the contact layer 16 and outer layer 18 may be restricted by stitching (not shown) which partially extends around the perimeter of the sole member. Other methods of attaching and retaining the sole member are contemplated within the scope of the present invention, including, for example, stitching, RF welding, heat lamination and solvent bonding.

Referring now to FIGS. 2 and 3, hook fasteners 56, 58 are provided for securing the wrapped cuff 12 around a foot F, and are positioned on the outer layer 18 of the cuff. Hook fastener 56 is mounted to strap portion 19b of outer layer 18 of foot cuff 12 while hook fastener 58 is mounted on a surface of outer layer 18. In use, when strap portions 19a, 19b are wrapped about foot F, hook element 56 engages outer layer 18 to facilitate mounting of foot cuff 12 to foot F. An identification tab (not shown) may also be included for providing information such as the model number and manufacturer name. Hook fasteners 56, 58 may have tabs (not shown) without fastening material thereon to provide convenient gripping locations on the hook fasteners to thereby allow the practitioner to easily remove the hooks from the outer face 18b of outer layer 18.

The use and operation of the foot cuff 12 for applying compression therapy to the wearer's foot is generally known in the art and will not be described herein. It is noted, however, that the ability of the inflatable member 14 to move or shift relative to and independent of the inner contact layer 16, in particular, reduces or prevents distortion of the inner contact layer because of the force being applied to the contact layer by the inflatable member.

It is understood that the above-described features of the present disclosure have similar applicability to other types of compression devices besides foot cuffs. For example and without limitation, the above-described features may be equally applicable to a leg compression sleeve such as the one generally indicated at reference numeral 112 in FIG. 5. As will become apparent, the pertinent components and structures of the leg compression sleeve 112 are similar to the corresponding components and structures of the foot cuff 12 of the above-described embodiment, and therefore, like components and structures are indicated by corresponding reference numerals plus 100. The leg compression sleeve of the illustrated embodiment includes overlying inner and outer layers 116, 118, respective, and separate, individual inflatable members 114 disposed between the inner and outer layers. The inner and outer layers 116, 118 are secured together along a line 117, although it is understood that the layers may be releasably secured together as taught by the previous embodiment.

As is apparent from the Figures, the main difference between the illustrated leg compression sleeve 112 and the illustrated foot cuff 12 in FIGS. 1-4 besides the size and shape of the sleeve is that the compression sleeve includes three inflatable members 114 spaced apart from one another along an axis of the sleeve. Like the inflatable member 14 of the above-described foot cuff 12, the inflatable members 114 of the compression sleeve 112 are configured for moving independently relative to the inner and outer layers 116, 118, respectively. Each of the inflatable members 114 may incorporate one or more of the features and alternatives described above with respect to the inflatable member 14 for the foot cuff 12. In general, each inflatable member 114 includes inner and outer inflatable layers secured together to define an inflatable chamber 127 within the periphery of the sealing line. Each inflatable member 114 has a securement margin 138 extending beyond the periphery of the bladder sealing line. A plurality of loop components 140 extend through slits 142 (broadly, openings) in the securement margin 138 of a respective inflatable member 114. The loop components 140 are secured to the outer layer 118. As will be understood by those having ordinary skill in the art, the other features and alternate embodiments the foot cuff 12 described, including the ways in which the inflatable bladder 14 is secured to either the outer layer 18 or the inner layer 16, may apply equally to the leg compression sleeve 112 of the present embodiment. Moreover, the illustrated leg compression sleeve 112 has a sealing line 117 extending around the perimeter of the sleeve to secure the inner layer 116 to the outer layer 118. It is understood that the inner layer 116 and the inner layer 118 may be releasably secured to one another, such as taught by the previous cuff embodiment 12. It is also understood that the above-described features of the foot cuff 12 may apply equally to other types of compression therapy devices.

The use and operation of the leg compression sleeve 112 is generally known in the art and therefore will not be described herein.

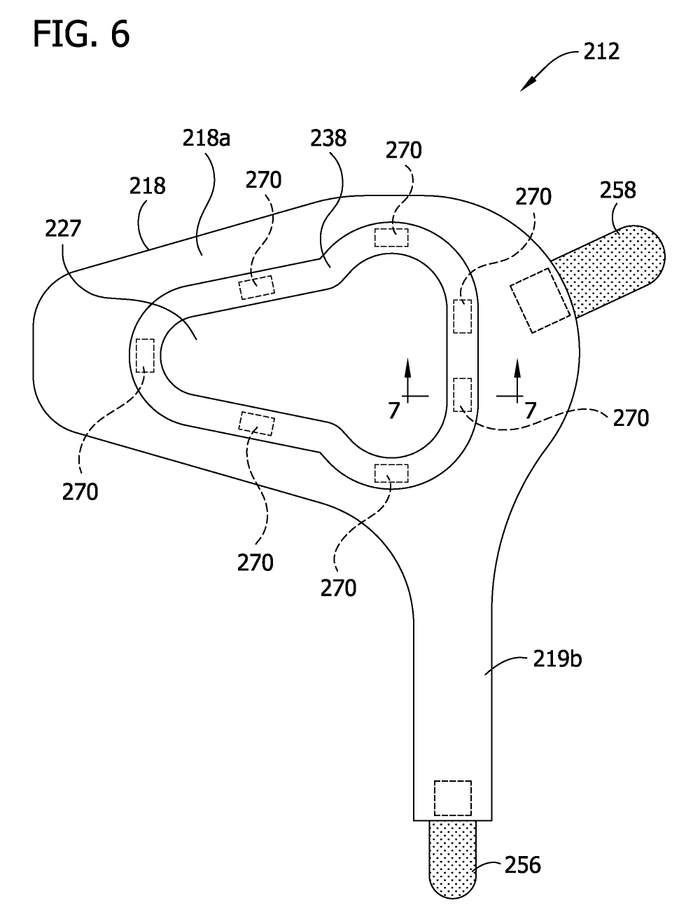

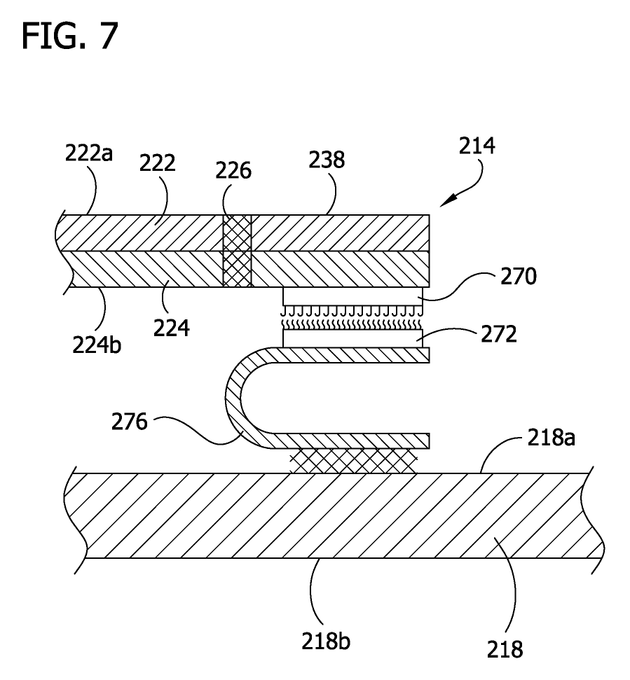

Referring now to FIGS. 6 and 7, yet another embodiment of a compression foot cuff is generally indicated at 212. The foot cuff of the present embodiment is similar to the foot cuff 12 in FIGS. 1-4, with the main difference being the manner in which the inflatable member 212 is attached to an outer layer 218. Because the present foot cuff 212 is similar to the foot cuff 12 in FIGS. 1-4, except for the manner in which the inflatable member 214 is attached to the outer layer 218, like components are indicated by corresponding reference numerals plus 200 throughout the drawings.

Referring to the present embodiment, a securement margin 238 of the inflatable member 214 of the foot cuff 212 does not have slits or openings receiving loop members or straps. Instead, first fasteners 270 secured to the securement margin 238 of the inflatable member 214 are spaced apart from one another around the periphery of the inflatable chamber 227 and are releasably securable (i.e., mateable) to second fasteners 272 secured to the outer layer 218. Accordingly, the inflatable member 214 can be removed from the mounting layer and from the cuff, as explained above with respect to the previous embodiment, so that it can be reused with another cuff. Exemplary types of releasable fasteners for use with this embodiment include, without limitation, hook and loop fasteners (illustrated), mateable snap-fasteners and other mechanical fasteners. Each first fastener component 270 of the illustrated embodiment is secured to the outer side (i.e., the outer face 224b of the outer layer 224) of the inflatable member 214. In the illustrated embodiment, each second fastener 272 is mounted on free end margins of folded straps 276 (broadly, extension members) secured to the outer layer 218. In the illustrated embodiment, the inflatable member 214 also moves independently relative to the inner contact layer 216 because the contact layer is secured around the perimeter of the outer layer 218 and not to the inflatable member to generally envelop or capture the inflatable member. In one example given without limitation, the straps 276 may be stretchable, preferably resiliently stretchable, to allow movement of the inflatable member 214 independently relative to the outer layer 218. In another example given without limitation, the straps 276 may non-stretchable but may be sized, i.e., have a suitable length, to have a suitable amount of slack that allows movement of the inflatable member 14 independently relative to the outer layer 218.

It is understood that the first fasteners 270 may be mounted on straps, and the second fasteners 272 may be secured directly to the outer layer 218, or both fasteners may be mounted on straps. It is also understood that the first and second fasteners 270, 272 may be secured directly to the respective inflatable member 214 and outer layer 218, without the need for a strap or other type of extension member. Alternatively, the securement margin 238 of the inflatable member may be fixedly (i.e., non-releasably) secured to the outer layer 218 or the inner layer 216, such as by welding, adhesive or in other ways. In these embodiments where the securement margin 238 is fixedly or releasably secured to the outer (or inner) layer 218 without an intervening strap or other extension member, it is preferable that a stretchability of the inflatable member 214, and more particularly a stretchability of the inflatable member at the perimeter margin 238, allows for satisfactory movement with respect to the inner and outer layers 216, 218.

Although not illustrated, it is understood that the features of the second embodiment of the foot cuff 212 may be equally applicable to and incorporated in other types of compression therapy devices.

It will be understood that numerous modifications and changes in form and detail may be made to the embodiments of the present disclosure. It is contemplated that numerous other configuration of the compression apparatus and geometries and orientation of the inflatable member may be used, and the material of the cuff and/or inflatable member may be selected from numerous materials other than those specifically disclosed. Therefore, the above description should not be construed as limiting the disclosed compression apparatus but merely as exemplifications of embodiments thereof. Those skilled in the art will envision numerous modifications within the scope of the present disclosure as defined by the claims appended hereto.

What is claimed is:

1. A compression device for applying compressive force to a part of a wearer's body comprising
an envelope comprising a mounting layer and a non-mounting layer disposed in generally opposing relationship to one another and being secured to one another to define an interior space within a periphery of the envelope,
an inflatable member disposed within the spaced defined by the interior space of the envelope, said inflatable member comprising layers in generally opposing relationship with one another, the layers being secured to one another to define an inflatable chamber, at least one of the layers extending beyond the periphery of the chamber to define a perimeter margin of the inflatable member,
the perimeter margin of the inflatable member being secured to the mounting layer of the envelope member to mount the inflatable member on the mounting layer,
the inflatable member being free from direct securement to the non-mounting layer to allow the inflatable member to move independently relative to the non-mounting layer.

2. A compression device as set forth in claim 1 further comprising
at least one securement hole extending through the perimeter margin of the inflatable member, and
at least one securement loop extending through said at least one securement hole so that the loop is secured to the inflatable member, the securement loop being secured to the mounting layer to thereby secure the perimeter edge margin of the inflatable member to the mounting layer.

3. A compression device as set forth in claim 2 wherein the securement loop is adapted to be opened and closed to releasably secure the loop to the inflatable member.

4. A compression device as set forth in claim 3 wherein the non-mounting layer and the mounting layer are releasably secured to one another to allow access to the interior space.

5. A compression device 3 wherein the securement loop comprises a strap having a first end margin secured to the mounting layer and a second end margin releasably secured to one of the mounting layer and the first end margin of the loop.

6. A compression device as set forth in claim 5 wherein first end margin of the strap includes a first connector and the second end margin of the strap includes a second connector releasably mateable to the first connector so that the second margin of the strap is releasably secured to the first end margin of the strap.

7. A compression device as set forth in claim 6 wherein the first connector comprises one of a hook fastener and a loop fastener and the second connector comprises the other one of a hook fastener and a loop fastener.

8. A compression device as set forth in claim 7 wherein the first end margin of the strap is fixedly secured to the mounting layer.

9. A compression device as set forth in claim 1 further comprising
a plurality of securement holes extending through the perimeter margin of the inflatable member, and
a plurality of securement loops extending through the securement holes so that the loops are secured to the inflatable member, the securement loops being secured to the mounting layer to thereby secure the perimeter edge margin of the inflatable member to the mounting layer.

10. A compression device as set forth in claim 8 wherein the plurality of securement holes are spaced apart along the perimeter margin of the inflatable member.

11. A compression device as set forth in claim 2 wherein the securement loop is elastically stretchable to allow the inflatable member to move independent of the mounting layer.

12. A compression device as set forth in claim 1 wherein the securement loop is substantially non-stretchable.

13. A compression device as set forth in claim 12 wherein the securement hole and the securement loop are sized and shaped to substantially fix the position of the inflatable member with respect to the mounting layer.

14. A compression device as set forth in claim 12 wherein at least one of the securement hole and the securement loop are sized and shaped to allow the inflatable member to move with respect to the mounting layer.

15. A compression device as set forth in claim 1 further comprising
at least one first fastener secured to the perimeter margin of the inflatable member, and
at least one second fastener secured to the mounting layer, said at least one second fastener being releasably mateable with said at least one first fastener to thereby releasably secure the perimeter margin of the inflatable member to the mounting layer.

16. A compression device as set forth in claim 15 further comprising at least one tether secured to the mounting layer, said at least one second fastener disposed on a free end margin of the tether to thereby secure the second fastener to the mounting layer.

17. A compression device as set forth in claim 1 wherein said non-mounting layer comprises an inner contact layer for contact with the wearer's skin during use, and wherein said mounting layer is an outer layer.

18. A compression device as set forth in claim 1 further comprising low-friction material at an interface of the inflatable member and the non-mounting layer to facilitate relative sliding of the inflatable member and non-mounting layer.

19. A compression device for applying compressive force to a part of a wearer's body comprising
at least one inflatable member comprising inner and outer layers each having respective inner and outer faces, the inner face of the outer layer being in a generally opposing relationship with the outer face of the inner layer, the inner and outer layers being secured to one another to define an inflatable chamber,
a mounting layer in generally opposing relationship with one of said outer face of the outer layer and said inner face of said inner layer,
a fastening mechanism mounting said at least one inflatable member to the mounting layer while allowing said at least one inflatable member to move independently relative to the mounting layer.

20. A compression device as set forth in claim 19 wherein,
at least one of the outer and inner layers includes a perimeter margin extending beyond a periphery of the chamber,
at least one securement hole extends through the perimeter edge margin of the inflatable member, and
at least one securement loop extends through said at least one securement hole so that the loop is secured to the inflatable member, the securement loop being secured to the mounting layer to thereby secure the perimeter margin of the inflatable member to the mounting layer.

21. A compression device as set forth in claim 19 wherein,
at least one of the outer and inner layers includes a perimeter margin extending beyond the periphery of the chamber sealing line,
at least one first fastener is secured to the perimeter margin of the inflatable member, and
at least one second fastener is secured to the mounting layer, said at least one second fastener being releasably mateable with said at least one first fastener to thereby releasably secure the perimeter margin of the inflatable member to the mounting layer.

22. A compression device as set forth in claim 19 further comprising a second layer secured to the mounting layer so as to form an envelope defining an interior space in which the inflatable member is enclosed.

23. A compression device as set forth in claim 22 wherein the second layer constitutes a non-mounting layer, wherein the inflatable member is not directly secured to the second layer.

24. A compression device as set forth in claim 22 wherein the second layer constitutes a second mounting layer, wherein said fastening mechanism mounts said at least one inflatable member to the second layer while allowing said at least one inflatable member to move independently relative to the second layer.
